(19) United States
(12) Patent Application Publication
(10) Pub. No.: US 2010/0003235 A1
(43) Pub. Date: Jan. 7, 2010

(54) ORAL FORMULATIONS FOR ENTERIC DISORDERS AND/OR REHYDRATION

(76) Inventors: Frank E. Hagie, Elk Grove, CA (US); Delia Bethell, Sacramento, CA (US); Ning Huang, Davis, CA (US)

Correspondence Address: ARENT FOX LLP, 1050 CONNECTICUT AVENUE, N.W., SUITE 400, WASHINGTON, DC, 20036, US

(21) Appl. No.: 12/088,631

(22) PCT Filed: Sep. 28, 2006
(86) PCT No.: PCT/US06/37720

§ 371 Date: Sep. 9, 2009

Related U.S. Application Data
(60) Provisional application No. 60/721,105, filed on Sep. 28, 2005.

(51) Int. Cl. A61K 38/00; A61P 1/00; A61K 38/40; A61K 38/47

(52) U.S. Cl. 424/94.61; 514/2; 514/6

(57) ABSTRACT

The present invention relates, generally, to oral formulations including one or more recombinantly-produced human milk proteins. The formulations of the present invention may be used to prevent the onset of diarrhea in patients who have been or will be exposed to one or more agents known to cause diarrhea, and to prevent the recurrence of diarrhea in a patient recovering therefrom. The formulations may also be used in the treatment of inflammatory bowel disease, including Crohn's disease and ulcerative colitis. The formulations may also be beneficially administered in accordance with methods for promoting the development of healthy intestinal flora in a human patient.

### BACKGROUND OF THE INVENTION

1. Field of the Invention

The present invention relates to the production and use of antimicrobials as components of an oral formulation for preventing and treating enteric disorders caused by a microbial organism or via other sources. The oral formulation may be used for the treatment and/or prevention of various intestinal diseases and conditions, for the control of intestinal pathogens, and for the rehydration of individuals with diarrhea.

2. Background of the Invention

Morbidity and mortality of children under the age of five due to diarrheal diseases remain a major health challenge in developing nations, as well as in lower socioeconomic groups in the developed world. It was not until the 1950s that diarrheal diseases were identified as a major cause of morbidity and mortality in children less than five years of age. The World Health Organization (WHO) reported approximately 1.5 million children under the age of five died from diarrheal disease in 2000.

Developed in the early 1970s, the WHO/UNICEF recommended a single formulation of glucose-based oral rehydration solution (ORS) (salt/sodium, sugar/carbohydrate and water) to prevent or treat dehydration from diarrhea irrespective of the cause or age group affected. The application of ORS has saved millions of children's lives worldwide. Today, ORS is considered one of the most significant medical advances of the 20th century. It has become the cornerstone of treatment for children with diarrhea and associated dehydration. Its use in treating diarrhea-related dehydration has increased from less than 15% of cases in 1984 to 40% in 1993.

This initial ORS formula was based on stool sodium levels in cholera patients and was designed to treat dehydration, not diarrhea. Consequently, although the hydration state was maintained, there was no decrease in the duration of the diarrhea episode. Thus controversy about the ideal composition of ORS and its suitability across all types of diarrhea: cholera and non-cholera, malnourished and well-nourished, children and adults, and children and infants, has been strong.

Scientific research has continued to develop new formulations of ORS with the key characteristics that it should be inexpensive, safe, effective, stable during prolonged storage, and that it have anti-diarrheal action (reduced volume and duration) as well as rehydration. One of the first areas of modification was in the source and form of the carbohydrate (glucose). In many cultures rice and rice water have been used as a home remedy for diarrhea. The replacement of the glucose with boiled rice, rice powder, rice flour or syrup was first tested in early 1980 and resulted in a decrease in stool output and diarrhea duration in cholera patients. A number of clinical and field studies followed comparing rice-based ORS with recommended WHO glucose-based ORS, and in all studies rice-based ORSs showed reduced stool output, decreased duration of diarrhea, and fewer unscheduled interventions with intravenous fluids.

A meta-analysis of the clinical trials using rice and other complex carbohydrates as a substitute for glucose in ORS suggests seven possible factors explaining the advantage of complex carbohydrates over glucose: 1) increased substrate availability, 2) a kinetic advantage for glucose absorption by glucose polymer, 3) differential handling of glucose monomer and polymer by the small intestine, 4) low osmolarity, 5) separate effect of peptides and amino acids on solute-linked sodium absorption, 6) an anti-secretary moiety in rice and 7) enhanced mucosal repair and regeneration by luminal nutrients. In 1994 a joint WHO/International Centre for Diarrhea Disease Research, Bangladesh (ICDDR, B) Consultative Meeting was held in Dhaka to review the results of 22 clinical trials using rice-based ORS. Although the rice-based ORS was superior for adults and children with cholera, it was not superior for children with acute non-cholera diarrhea. In addition, the group concluded that the increased cost of the rice-based material did not justify changing the WHO recommended formulation. This cost conclusion has been disputed based on the decrease in duration of diarrhea requiring shorter treatment and less ORS use, thus making the cost equivalent. It has also been pointed out that some developing countries do not have a ready supply of glucose.

The improved action of the rice-based ORS and its reduced osmolarity led to the investigation of new glucose-based formulations with reduced osmolarity. The reduced osmolarity ORS was tested using several variations of sodium and glucose concentrations. In a review of 15 randomized trials enrolling 2,397 patients, the reduced osmolarity rehydration solution was associated with reduced need for unscheduled intravenous infusions, lower stool volume, and less vomiting than the standard WHO formulation. In 2001, an expert consultation on oral rehydration solution formulation was held in New York to review the data comparing the single glucose-based formulation recommended by WHO/UNICEF with the lower osmolarity formulation containing less sodium and glucose. Following that meeting, WHO/UNICEF changed the single-formula recommendation to a low osmolarity formulation.

Diarrhea may be caused by a temporary problem, like an infection, or a chronic problem, like an intestinal disease. Diarrheal illnesses may be classified as osmotic (due to an increase in the osmotic load presented to the intestinal lumen, either through excessive intake or diminished absorption), inflammatory or mucosal (when the mucosal lining of the intestine is inflamed), secretory (when increased secretory activity occurs), and motile (caused by intestinal motility disorders). A few of the more common causes of diarrhea are:

(1) Bacterial infections. Several types of bacteria, consumed through contaminated food or water, can cause diarrhea. Common culprits include Clostridium, Campylobacter, Salmonella, Shigella, and Escherichia coli.

(2) Viral infections. Many viruses cause diarrhea, including rotavirus, Norwalk virus, cytomegalovirus, herpes simplex virus, and viral hepatitis.

(3) Food intolerances. Some people are unable to digest some component of food, such as lactose, the sugar found in milk.

(4) Parasites. Parasites can enter the body through food or water and settle in the digestive system. Parasites that cause diarrhea include Giardia lamblia, Entamoeba histolytica, and Cryptosporidium.

(5) Reaction to medicines, such as antibiotics, blood pressure medications, and antacids containing magnesium.

(6) Intestinal diseases, like inflammatory bowel disease (IBD) or celiac disease.

(7) Functional bowel disorders, such as irritable bowel syndrome, in which the intestines do not work normally.

Clostridium difficile is a gram-positive, spore-forming, anaerobic organism which has become one of the leading causes of nosocomial (i.e., hospital acquired) diarrhea. The organism was initially identified in the fecal flora of healthy newborn infants. The organism colonizes up to 50% of health infants, but is rarely found in normal, non-hospitalized adults. It was not until the 1970s that C. difficile was “rediscovered” as the agent associated with diarrhea and colitis following the use of broad spectrum antibiotics. Today, C. difficile is recognized as a major cause of diarrhea in hospitalized adults in developed countries.

The risk of community-acquired C. difficile is very low, but the risk of colonization and overt disease (diarrhea to pseudomembranous colitis) increases in direct relationship to the length of a hospital stay and the use of therapy with antibiotics. The use of broad spectrum antimicrobial therapy disturbs the normal flora of the gut and allows the colonization with C. difficile. This risk was previously estimated at greater than 20% for those hospitalized for over a week, however, with a new monoclonal antibody based rapid ELISA for Toxin A and B; it is possible that this rate may be doubled.

The treatments of choice for C. difficile are discontinuation of the causative broad spectrum antibiotic when possible and dosing with metronidazole or vancomycin. Although most patients respond to specific therapy there are issues of drug intolerance and development resistance. In addition 5% to 30% of patients will experience relapse and recurrent infection.

In 1998, three unresolved problems with respect to C. difficile were identified. First was the lack of a sensitive and specific test. This problem has been addressed with the development of a more sensitive and specific ELISA test for toxin A and B. Second, was the need for improvement in hospital control barrier methods. One area suggested was the control of antimicrobial use patterns in hospitals. In addition, strong adherence to hand washing and other standard infection control protocols can be effective. The third area was the recurrence of the disease. The fact that the treatment for a condition brought about by antibiotic use is another antibiotic may result in the recurrence of disease in a continuously hospitalized patient.

IBD is a lifelong condition with significant morbidity and impact on quality-of-life. Twenty to thirty percent of the diagnoses are in children. The etiology of IBD is not entirely known but genetic, microbial, and immune factors play important roles. Traditional immunomodulatory therapy, including steroids, can have a significant negative effect on growth and development.

Breast-fed children have a lower incidence of diarrhea, as well as other infections. Breast milk contains a number of innate antimicrobial proteins that may play a role in the reduction of diarrhea and in the promotion of colonization of the gastrointestinal tract with healthy or commensal microflora that act as a deterrent to ongoing and future diarrhea episodes. In vitro data suggest that the milk proteins lactoferrin (LF) and lysozyme (LZ) play an important part in the antimicrobial role of breast milk. LF and LZ individually and in combination have demonstrated antimicrobial activity against a wide spectrum of bacteria, viruses, parasites, and fungi. LF also has immunomodulatory properties, up-regulating the anti-inflammatory cytokines and down-regulating the pro-inflammatory cytokines in the intestinal tract.

There is a current market absence of a source of safe and cost effective bioactive human milk proteins that exhibit this effect. Requirements for such proteins include the following:
a. Proteins must be bioactive.
b. Proteins must be produced and isolated in a cost-effective system.
c. Abundant supply of these bioactive milk proteins is necessary.
d. Produced in a host that is essentially devoid of human pathogens and/or toxins. Donated human breast milk includes pathogens, as does production of these molecules in transgenic bacteria, yeast, fungi, animals and cell culture.
e. Capable of retaining bioactivity in multiple formulations.
f. Stable in storage as formulated.

The present invention addresses these requirements.

There remains a need to prevent and treat intestinal diseases using a more effective formulation, for example complex carbohydrates and antimicrobial proteins. Further, if the formulation of complex carbohydrates or antimicrobial proteins is in concentrations that are similar to those found in the innate immune system, such a treatment can have synergistic beneficial effects such as the control of intestinal organisms such as C. difficile. Such treatment can be alone or in conjunction with standard treatments with antibiotics.

### SUMMARY OF THE INVENTION

One aspect of the invention is an oral formulation that is used to prevent or treat intestinal diseases or conditions. Examples of intestinal diseases or conditions include diarrhea, Crohn's disease, and ulcerative colitis.

Another aspect of the invention is an oral formulation that is helpful in the control of intestinal pathogens. Examples include Clostridium, Campylobacter, Salmonella, Shigella, and Escherichia coli.

An additional aspect of the invention is an oral formulation that is helpful in promoting the growth of beneficial intestinal flora.

A further aspect of the invention is an oral formulation including rhLF in an amount from about 0.5 to about 5.0 g/L; and rhLZ in an amount from about 0.1 to about 1.0 g/L. Preferably, the oral formulation has an osmolality of from about 200 to about 310 mOsm/L. The oral formulation preferably delivers antimicrobial activity along with rehydration and feeding to provide a major improvement in reducing the duration and/or severity of intestinal diseases or conditions, and enhancing the rate of recovery.

Another aspect of the invention comprises a method for the production of an oral formulation, the method comprising incorporating antimicrobials into the formulation, wherein the formulation confers at least one of the following benefits: improving rehydration, preventing the onset or recurrence of diarrhea, reducing diarrhea duration and/or volume, controlling intestinal pathogens such as C. difficile, and promoting the growth of beneficial intestinal flora.

Other novel features and advantages of the present invention will become apparent to those skilled in the art upon examination of the following or upon learning by practice of the invention.

### BRIEF DESCRIPTION OF THE FIGURES

The invention will be described in further detail with reference to the accompanying Figures.

FIG. 1 shows the difference in the duration of diarrhea between an oral solution prepared according to the present invention and a standard ORS;

FIG. 2 shows the difference in the volume of diarrhea between an oral solution prepared according to the present invention and a standard ORS;

FIG. 3 shows the percent of patients that did not reach 48 hours with solid stool using an oral solution prepared according to the present invention compared with a standard ORS;

FIG. 4 shows the percent of patients with relapse using an oral solution prepared according to the present invention compared with a standard ORS;

FIG. 5 shows the volume of solution consumed (oral solution prepared according to the present invention compared with a standard ORS);

FIG. 6 shows a comparison of E. coli colony formation in media with and without 1 mg/ml rhLF showing reduction of colony in treatment with rhLF;

FIG. 7 shows a line graph of lactoferrin inhibiting bacterial cell growth as measured by optical density at wavelength (A630). The three treatments are control (media only), native (contain native human lactoferrin) and recombinant (contain recombinant human lactoferrin);

FIG. 8 shows a comparison of E. coli colony formation in media with and without 20 ug/ml rhLZ showing reduction of colony in treatment with rhLZ; and

FIG. 9 shows a line graph of colony forming units of E. coli from three treatments, buffer only (black line with white square box), buffer plus native human lysozyme (red line) and buffer plus recombinant human lysozyme (green line).

### DETAILED DESCRIPTION OF THE INVENTION

The term “stably transformed” with reference to a plant cell means the plant cell has a non-native (heterologous) nucleic acid sequence integrated into its genome which is maintained through two or more generations.

By “host cell” is meant a cell containing a vector and supporting the replication and/or transcription and/or expression of the heterologous nucleic acid sequence. Preferably, according to the invention, the host cell is a plant cell, most preferably a monocot plant cell, such as rice or barley. Other host cells may be used as secondary hosts, including bacterial, yeast, insect, amphibian or mammalian cells, to move DNA to a desired plant host cell.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The term “mature plant” refers to a fully differentiated plant.

The term “seed product” includes, but is not limited to, seed fractions such as de-hulled whole seed, flour (seed that has been de-hulled by milling and ground into a powder) a seed extract, preferably a protein extract (where the protein fraction of the flour has been separated from the carbohydrate fraction), malt (including malt extract or malt syrup) and/or a purified protein fraction derived from the transgenic grain.

The term “biological activity” refers to any biological activity typically attributed to that protein by those skilled in the art.

“Monocot seed components” refers to carbohydrate, protein, and lipid components extractable from monocot seeds, typically mature monocot seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Maturation-specific protein promoter” refers to a promoter exhibiting substantially up-regulated activity (greater than 25%) during seed maturation.

“Heterologous DNA” refers to DNA which has been introduced into plant cells from another source, or which is from a plant source, including the same plant source, but which is under the control of a promoter that does not normally regulate expression of the heterologous DNA.

“Heterologous protein” is a protein encoded by a heterologous DNA. The proteins include, but are not limited to, antimicrobial proteins and peptides, lactoferrin (which may be substituted with lactoferricin), lysozyme, haptocorin, lactahedrin, defensin, cathelicidins, and lactoperoxidase.

As used herein, the terms “native” or “wild-type” relative to a given cell, polypeptide, nucleic acid, trait or phenotype, refers to the form in which that is typically found in nature.

As used herein, the term “purifying” is used interchangeably with the term “isolating” and generally refers to any separation of a particular component from one or more other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results have less of the other components (“contaminating components”) than before such purifying or isolating step(s).

As used herein, the terms “transformed” or “transgenic” with reference to a host cell means the host cell contains a non-native or heterologous or introduced nucleic acid sequence that is absent from the native host cell. Further, “stably transformed” in the context of the present invention means that the introduced nucleic acid sequence is maintained through two or more generations of the host, which is preferably (but not necessarily) due to integration of the introduced sequence into the host genome.

“ORS” refers to a solution that is used to prevent or correct dehydration. An ORS typically, but not necessarily, contains a mixture of salt, sugar, potassium, and other minerals to help replace body fluids lost to disease or condition.

“An oral formulation ingredient” includes one or more of proteins, peptides, hormones, carbohydrates, amino acids, lipids, vitamins, organic and inorganic salts.

“An oral formulation active ingredient” or “oral formulation component” refers to any antimicrobials, proteins and non-proteins, recombinant and non-recombinant, added to or supplemented to an oral formulation.

“An oral formulation supplement” refers to a combination of one or multiple oral formulation components with or without other ingredients for addition to an oral formulation.

“Antimicrobial” refers to a group of chemicals that have the function of antibacterial, antifungal, antiprotozoal and/or antiviral agents.

“Recombinant proteins” refers to heterologous proteins produced using recombinant DNA technology.

“Intestinal diseases or conditions” include diarrhea (from any cause, for example any of the individual causes listed previously), Crohn's disease, diverticulosis and diverticulitis, gastric cancer, gastritis, ulcerative colitis, peptic ulcer, intestinal ulcer, gastric ulcer, duodenal ulcer, irritable bowel disease, irritable bowel syndrome, constipation, infection of intestinal pathogens, and hemorrhoids.

The addition of human lactoferrin and lysozyme to an oral solution results in a reduced duration of diarrheal disease and enhanced rate of recovery of the intestinal mucosa.

Lactoferrin has been shown to have in vitro antiviral effects against rotavirus infection of HT-29 cells, an enterocyte-like cell line. The antiviral mechanism of action appears to be two-fold. The lactoferrin binds directly to the virus particle and prevents it from binding to the target cell. It also has an inhibitory effect on viral antigen synthesis and viral yield when cells are exposed to lactoferrin after the attachment step. This second mechanism requires uptake of lactoferrin by specific cell receptors. Another significant observation was that the activity of bovine lactoferrin against rotavirus was increased following desialylation. It is of note that recombinant human lactoferrin produced in rice has no sialic acid. Although not as commonly detected, adenovirus can also be associated with diarrhea. Lactoferrin is able to inhibit adenovirus infection of cells by binding to the glycosaminoglycan receptors and blocking viral attachment to the cell membrane.

Lactoferrin also has been shown to actively impair Shigella virulence. Epidemiologic studies in Bangladesh have demonstrated that breast-fed infants have reduced incidence and severity of Shigella infection. In vitro studies suggest that lactoferrin acts at the surface of the bacteria to cause the invasion antigens to be released and become more susceptible to a protease. These same studies demonstrated that the loss of invasion antigens was not associated with iron saturation or the N-terminal cationic peptide of lactoferrin.

The anti-inflammatory and immunomodulatory effects of human lactoferrin make this “food” substance a good candidate for nutritional support for children and/or adults with intestinal diseases or conditions, such as mild to moderate IBD. The opportunity to incorporate an adjunct to IBD treatment and/or control of intestinal pathogens, such as C. difficile, using a protein of the innate immune system in conjunction with standard antimicrobial treatment is available through the use of lactoferrin. This protein is found in the epithelial secretions and is presumed to help with barrier protection functions. Lactoferrin is an iron binding glycoprotein of the transferrin family and is associated with anti-microbial properties and boosting of immunity at the mucosal level. Lactoferrin and specifically pepsin generated peptides from lactoferrin stimulate the growth of bifidobacteria. Lactoferrin has demonstrated the ability to modulate the inflammatory cytokine response in the intestine in the rat colitis model system. It has also reduced damage by indomethacin in healthy volunteers.

In one embodiment of the present invention, lactoferrin is produced through protein expression and purification in a rice grain based system. The lactoferrin so produced has undergone extensive testing to establish its substantial equivalence to the native protein purified from breast milk.

Another embodiment of the present invention comprises the use of lactoferrin for treatment, control or prevention of infection by intestinal pathogens. For example, lactoferrin may be used in hospitalized patients, to treat, control or prevent C. difficile infection. The natural protein, used at or above average breast milk levels (at least 1 mg/mL lactoferrin) may control or prevent pathogen colonization in subjects, especially hospitalized or long term care patients who require broad spectrum antimicrobial therapy. Preliminary data from in vitro studies demonstrates susceptibility of clinical isolates to recombinant human lactoferrin. In a method to treat or prevent pathogenic, preferably C. difficile, infection, the dosing regimen may be about 0.5 to 10 g, preferably about 2 to 8 g, most preferably about 3 g of lactoferrin every 24 hours for a period of time sufficient to treat or prevent the infection, with or without concomitant antibiotic treatment.

In a related embodiment, the use of lactoferrin, whether alone or in conjunction with lysozyme, may promote the development and maintenance of healthy gut flora. Examples of such probiotic bacteria may include members of the Lactobacillus, Bifidobacterium, Lactococcus, Enterococcus, Saccharomyces, and Acidophilus genuses, but are not limited to these.

Human lysozyme is a 1,4-β-N-acetylmuramidase. It enzymatically degrades a glycosidic linkage of peptidoglycan in the cell membrane of gram-positive bacteria. Acting alone, lysozyme lyses and kills several gram-positive microorganisms. Typically, the outer membrane of gram-negative bacteria protects them from lysozyme. In contrast, lactoferrin binds and alters the membrane of gram-negative bacteria. These two proteins found in breast milk and other mucosal secretions demonstrate bacteriostatic activity alone, but are able to act in a synergistic, bactericidal fashion when both are present. Lactoferrin alone has bacteriostatic properties. One mechanism of action of lactoferrin is through the sequestration of iron, depriving the microorganisms of an essential nutrient. Lactoferrin, however, also has an N-terminal region that is bactericidal and has lipopolysaccharide (LPS) binding activities. In studies on V. cholerae, lysozyme alone was inactive, lactoferrin alone was bacteriostatic and the combined proteins were bactericidal. Similar results were found with S. typhimurium and E. coli. Lactoferrin and lysozyme are effective against E. coli individually and in combination.

Human milk and lactoferrin also have been studied for anti-parasitic activity. Both have shown cidal activity against Giardia lamblia trophozoites. In the case of cryptosporidiosis, breast-feeding has been an effective deterrent. This could be both an antimicrobial role and an anti-inflammatory role through cytokine modulation.

Lactoferrin may have a positive role in protection of the intestinal mucosa during diarrhea. Studies in animals with oral administration of lactoferrin have demonstrated a protective effect against development of colitis via modulation of the immune system. This is accomplished by lactoferrin-induced increases in the anti-inflammatory cytokines IL-4 and IL-10 and the inhibition of release of the pro-inflammatory cytokines IL-6, TNF-α, and IL-1β. This protective mechanism may result in less damage and more rapid repair of gut mucosal tissue leading to normal permeability and growth. Lactoferrin also protected gut mucosa against the effects of bacterial lipopolysaccharide in mice. Supported by studies on lactoferrin as an anti-inflammatory and immunomodulatory protein, the use of human lactoferrin in the oral formulations of the present invention may be beneficial when administered either alone or in conjunction with known methods for treating inflammatory bowel disease, such as Crohn's disease or ulcerative colitis.

The purification of lactoferrin and lysozyme from human breast milk is not an economically feasible option. Expression of human lactoferrin and lysozyme in plants, preferably monocot plants such as rice, is an attractive approach, since rice is among the first foods recommended for introduction to infants. It has nutritional value and low allergenicity. After purification, any residual protein or carbohydrate from the rice introduce no risk and may be viewed as nutritionally sound. Since human lactoferrin and lysozyme are major parts of the diet of breast-fed infants, the addition of recombinant human lactoferrin and lysozyme to the diet will be an extension of their normal intake.

The oral formulation or ORS may advantageously contain 0.0001% to 10% by weight of antimicrobial proteins, preferably selected from the group consisting of lactoferrin, lysozyme, defensin, cathelicidins, and lactoperoxidase. Preferably, these proteins are provided in an amount of from about 0.001% to 1% by weight of the oral formulation or ORS. More preferably, if lactoferrin and/or lysozyme are utilized, they are present at least in the amount found in human breast milk.

Production of antimicrobial proteins and peptides, such as lactoferrin and lysozyme, in accordance with the present invention may be achieved using the teachings of U.S. patent application Ser. Nos. 10/077,381, 10/411,395, PCT/US2004/041083, and/or PCT/US2003/39107, each of which are hereby incorporated by reference in their entirety. The so-produced antimicrobial proteins and peptides may be utilized with or without further purification, and added to an oral formulation.

A preferred method of producing antimicrobial proteins and peptides in monocot plant seeds comprises the steps of:
(a) transforming a monocot plant cell with a chimeric gene comprising
   (i) a promoter from the gene of a maturation-specific monocot plant storage protein,
   (ii) a first DNA sequence, operably linked to said promoter, encoding a monocot plant seed-specific signal sequence capable of targeting a polypeptide linked thereto to a monocot plant seed endosperm cell, and
   (iii) a second DNA sequence, linked in translation frame with the first DNA sequence, encoding an antimicrobial protein and/or peptide, wherein the first DNA sequence and the second DNA sequence together encode a fusion protein comprising an N-terminal signal sequence and the antimicrobial protein and/or peptide;
(b) growing a monocot plant from the transformed monocot plant cell for a time sufficient to produce seeds containing the antimicrobial protein and/or peptide; and
(c) harvesting the seeds from the plant.

Preferably, the antimicrobial protein and/or peptide constitutes at least 3.0% of the total soluble protein in the seed product, or at least 0.1% of total seed weight.

The expression of recombinant human lactoferrin in cereal grains, such as rice and barley, provides a cost effective method for the production and isolation of lactoferrin. Human lactoferrin produced in rice has been shown to be substantially equivalent to human lactoferrin from breast milk. In addition, studies have demonstrated that the difference in the glycan pattern is highly unlikely to present an allergenicity risk. Supported by the studies on lactoferrin as an anti-microbial, anti-inflammatory and immunomodulatory protein, the use of human lactoferrin in gastrointestinal disease provides ideal applications. In addition to their use in treating, preventing, and reducing the impact of diarrhea in patients, the nutritional support products in accordance with the present invention may be administered to promote the development of healthy gut flora in patients.

An oral rehydration solution containing human lactoferrin in combination with a second breast milk protein, lysozyme, may be used for children with acute watery diarrhea. Human lactoferrin and/or lysozyme may also be beneficial in preventing diarrhea in long-term care geriatric patients. In another embodiment of the present invention, human lactoferrin and/or lysozyme can be used as nutritional support and prophylaxis in travelers' and military diarrhea.

The formulations of the present invention may be administered in any manner suitable to produce the desired effect—be it rehydration, treatment of intestinal diseases or conditions, prevention of the onset or recurrence of intestinal diseases or conditions, control of intestinal pathogens such as C. difficile, and promote the growth of commensal microflora in the intestinal tract. The formulations may be provided as solutions, in dry form (powder) for reconstitution, liquid concentrates, which can be added to water, juice, yogurt, etc., formulated as a nutrition bar, a capsule, a tablet, a wafer, etc.

### EXAMPLES

### Example 1

### Expression of Recombinant Human Lactoferrin

### A. An Expression Vector for Human Lactoferrin Expression in Transgenic Rice

The complete nucleotide sequence of human mammary gland lactoferrin was codon optimized and synthesized by Operon Technologies (CA, USA). Human milk lactoferrin gene (Genbank accession number: HSU07642) was re-synthesized with codons most frequently used in translation of rice seed proteins in order to obtain optimal level of expression. Although the numbers of codons changed accounted for 22.46% of the entire sequence, the amino acid composition remained identical to non-recombinant human lactoferrin. The plasmid containing the codon-optimized gene was called Lac-ger. Lac-ger was digested with SmaI/XhoI and the fragment containing the lactoferrin gene was cloned into pAPI141 that was partially digested with NaeI and completely digested with XhoI. For expression of hLF in rice seeds, the codon-optimized gene was operably linked to the rice endosperm-specific glutelin (Gt1) promoter and NOS terminator. The resulting plasmid was designated pAPI164.

### B. Production System

Rice variety Taipei 309 (Oryza sativa, Japonica) was selected as the production system for recombinant human lactoferrin (rhLF) and transgenic rice plants were eventually generated by the particle bombardment of embryogenic rice calli with the plasmid pAPI164 and a companion marker plasmid containing the hygromycin phosphotransferase gene as a selectable marker. Fully developed, fertile rice plants were obtained by this procedure.

### C. High Level Protein Expression of Recombinant Human Lactoferrin in Rice Grain

Expression of recombinant human lactoferrin was under the control of the seed maturation-specific promoter Gt1. The high level expression of recombinant human lactoferrin was evident. Total soluble proteins from mature rice seed extracts were run on Laemli gels and stained with Coomassie blue to visualize the proteins. An ˜80 kD recombinant lactoferrin protein was obtained in all transgenic lines as indicated by the stained gel. Expression levels of recombinant human lactoferrin corresponded to 0.5% by weight of seed. The stable expression of recombinant human lactoferrin was monitored for 10 generations. The expression level was maintained at around 5 g/kg of brown rice.

### Example 2

### Purification of Recombinant Human Lactoferrin

To prepare an oral rehydration solution supplemented with recombinant human lactoferrin, recombinant human lactoferrin was purified from rice flour. A transgenic rice line (164-12) expressing high levels of rhLF was selected. This line, now named as LF164, was planted two generations per year, alternating field planting in summer and greenhouse planting in winter. For protein purification, paddy rice expressing rhLF was de-hulled by using a de-huller (Rice Mill, PS-160, Rimac, Fla.), and then ground to flour (average particle size of 100 mesh) using a hammer mill (8WA, Schutte—Buffalo, N.Y.).

Protein extraction from transgenic flour was performed by mixing two kg of rice flour and 20 L of extraction buffer (0.02 M sodium phosphate pH 6.5 and 0.3 M sodium chloride) in a 50 L tank for 1 h. At the end of the mixing period, the suspension was allowed to settle overnight or centrifuged at 3750 rpm. In both cases, the supernatant was filtered through a plate and frame filter (Ertel Alsop, 8S, NY) using M-05 and M-70 cellulose/perlite-based filters (Ertel Alsop, NY), respectively.

The filtrate containing rhLF and other rice flour soluble proteins was loaded onto an ion exchange column for further purification. An INDEX 200/500 process column (Amersham Pharmacia Biotech, NJ) packed with SP-Sepharose fast flow (Amersham Pharmacia Biotech, NJ) was used. The column was used with linear flow rates of 150-200 cm/h. Packing, cleaning and testing of the packed-column performance was executed per manufacturer's instruction. The filtrate was loaded on the column at a linear velocity of 175 cm/h and washed with 0.02 M sodium phosphate buffer (pH 6.5) containing 0.3 M NaCl until the A₂₈₀ returned to baseline. Recombinant hLF was eluted using 20 mM sodium phosphate buffer (pH 6.5) containing 0.8 M NaCl. The washing and elution were performed at 200 cm/h and 150 cm/h, respectively.

A Centramate module (Pall Biopharmaceutical, MA) with 1 ft² 50 kDa polyethersulfone (Pall Biopharmaceutical, MA) membrane was used for concentration and desalting (infiltration) of eluted hLF. The filtration was performed at a cross flow rate of about 1.5 L/min and an average Tran membrane pressure of 10 psig. The eluted rhLF was concentrated and desalted to a final volume of 0.25 L and then lyophilized dry. Usually, about 3 grams of purified recombinant human lactoferrin was recovered from one kilogram of transgenic rice flour.

The recombinant human lactoferrin purified from rice flour was approximately 50% saturated with iron (partial-lactoferrin). The 50% saturated recombinant human lactoferrin was then made >90% iron saturated by iron up taking treatment, resulting in holo-lactoferrin and was made <10% iron saturated by acid treatment to remove bound iron resulting in apo-lactoferrin.

### Example 3

### Production and Purification of Recombinant Human Lysozyme

Recombinant human lysozyme is produced in the LZ159 rice variety derived from Oryza sativa, Japonica, Taipei 309. The rice is dehusked and milled to an average of 100 mesh flour using standard food industry procedures. Recombinant human lysozyme is extracted from ground rice flour using 0.02 M acetate buffer with 0.3 M NaCl pH 4.5. After 1.0 to 2.0 hours of extraction, the solid rice flour is separated from the liquid phase by centrifugation. The liquid phase, containing the soluble protein, is filtered and concentrated. At this point the product is a protein concentrate that is >10% lysozyme protein. The concentrate is further purified to an isolate using ion exchange chromatography. The concentrate is loaded on a column of SP Sepharose Big Bead Media, the column is washed and the bound lysozyme eluted with 0.8 M sodium chloride. The isolate is ultrafiltered to remove excess salt and concentrated prior to lyophilization. The isolate form of lysozyme is >80% pure lysozyme protein as measured by HPLC analysis.

### Example 4

### Comparison of ORS Containing Recombinant Proteins with Standard ORS

TABLE 1

| | Exemplified Rice- | Acceptable |
|---|---|---|
| Ingredients | ORS-LF/LZ | Range |
| Sodium (mmol/L) | 75 | 60-90 |
| Potassium (mmol/L) | 20 | 15-25 |
| Chloride (mmol/L) | 65 | 50-80 |
| Citrate (mmol/L) | 10 | 8-12 |
| Glucose (mmol/L) | — | <111 |
| Carbohydrate (g/L) | 41.0 | 25-50 |
| rhLF (g/L) | 1.0 | 0.5-5.0 |
| rhLZ(g/L) | 0.2 | 0.1-1.0 |
| Osmolality (mOsm/L) | 220-240 | 200-310 |

An ORS containing recombinant human lactoferrin (rhLF) and recombinant human lysozyme (rhLZ) in concentrations similar to those found in human breast milk (1 mg/mL lactoferrin and 0.2 mg/mL lysozyme) along with other components (as shown in Table 1) was compared with a standard ORS in a prospective double-blind study of children with acute watery diarrhea. A sachet of ORS was dissolved in 1 liter of water that had been boiled and cooled. Fresh ORS was prepared daily.

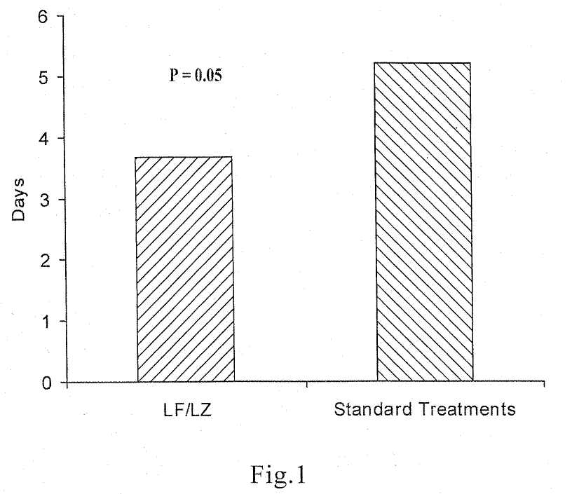

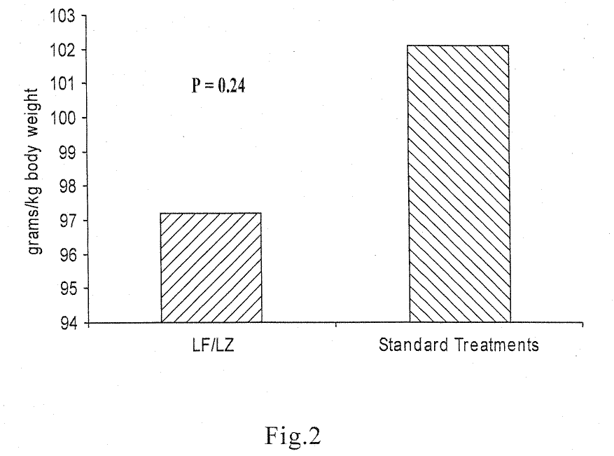

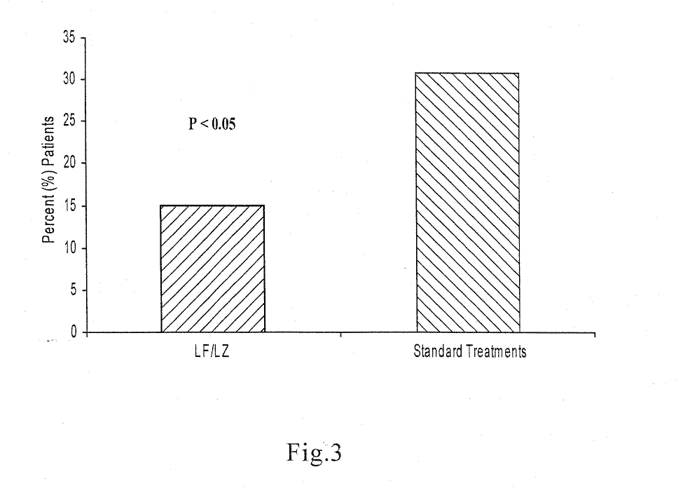

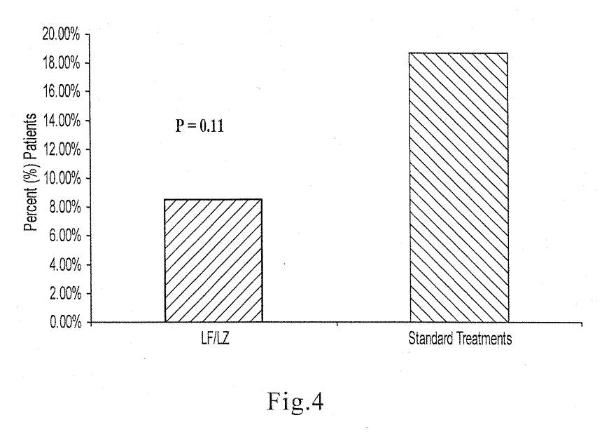

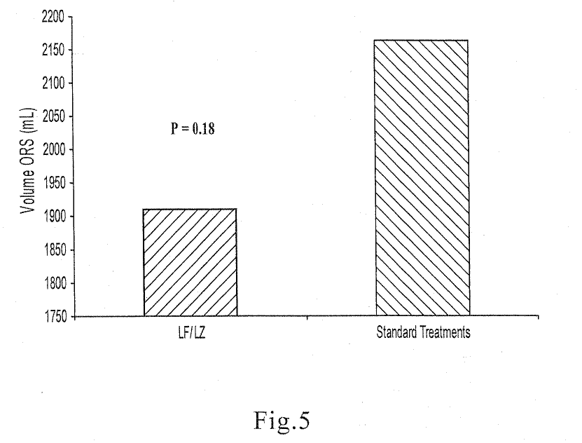

One hundred and forty children with watery acute diarrhea were given either an ORS according to the present invention or a standard ORS prepared according to WHO guidelines for 14 days. Treatment continued until the diarrhea ceased for two days or for 14 days post enrollment. The study indicated that the ORS containing lactoferrin and lysozyme was superior to the standard ORS as measured by decreased duration and volume of diarrhea (FIG. 1 to FIG. 5).

### Example 5

### Antimicrobial Effect of Recombinant Human Lactoferrin

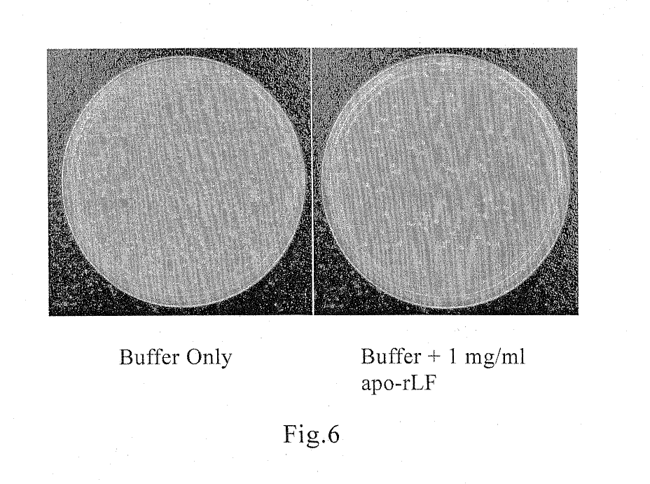

The antimicrobial activity of recombinant human lactoferrin was measured using E. coli as substrate. Cultured cells of E. coli K 2 were prepared from culture plates. About 10⁵ CFU of E. coli in 1 mL was mixed with 1 mg of rhLF; the control contained no lactoferrin. The mixture was incubated at 37° C. for 120 minutes with shaking at 250 rpm. Five μL of mixture was then plated. As can be seen in FIG. 6, there is marked reduction in colony forming units in the culture with added rhLF.

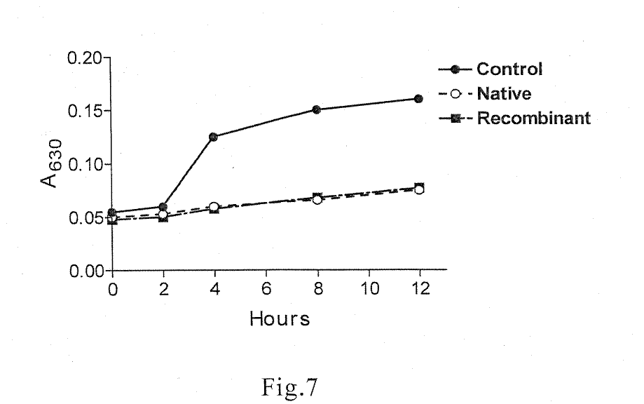

To compare the effect of rhLF and native hLF on E. coli growth, another experiment was carried out with three groups: negative control (growth media only), positive control (growth media with native hLF) and treatment (growth media with rhLF) As shown in FIG. 7, there was a marked increase of E. coli growth in growth media while E. coli growth was much slower in treatments with native and recombinant human lactoferrin indicating that recombinant human lactoferrin has the same effect as the native lactoferrin in controlling E. coli growth in culture media.

### Example 6

### Antimicrobial Effect of Recombinant Human Lysozyme

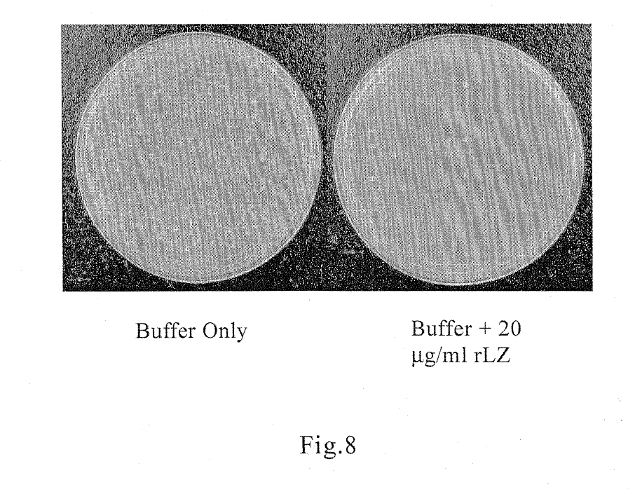

The antimicrobial activity of recombinant human lysozyme was measured using E. coli as substrate. Cultured cells of E. coli K 2 were prepared from culture plates. About 10⁵ CFU of E. coli in 1 mL was mixed with 20 μg of rhLZ; the control contained no lysozyme. The mixture was incubated at 37° C. for 120 minutes with shaking at 250 rpm. Five μL of mixture was then plated. As can be seen from FIG. 8, there is marked reduction in colony forming units in the culture with added rhLZ.

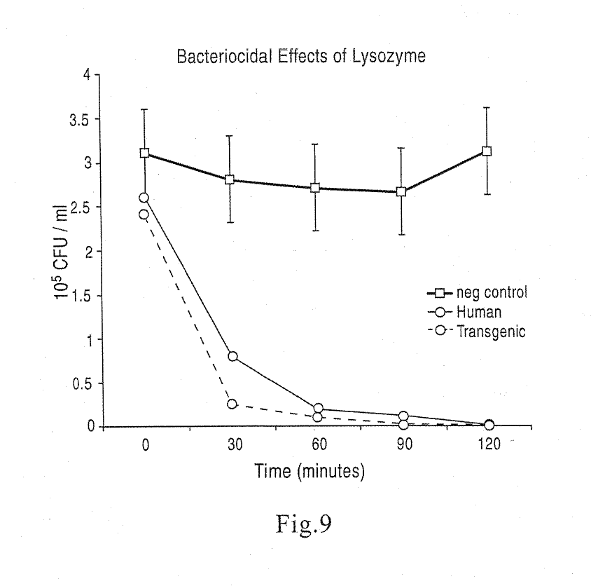

To compare the antibacterial effect of rhLZ and native hLZ, the same experiment was repeated with three groups: negative control (buffer only), positive control (buffer with native hLZ) and treatment (buffer with rhLZ) (FIG. 9). There was no reduction in colony forming units in the negative control while there was a significant decrease of E. coli in the positive control. At 60 min incubation time, colony forming units were reduced to 100 and approached zero at 120 min. Treatment with rhLZ had the same effect as the positive control, indicating that both rhLZ and hLZ have the bactericidal activity.

### Example 7

### Control of C. difficile with Lactoferrin In Vitro

This example was conducted to determine the activity of lactoferrin against C. difficile. The study consisted of in vitro susceptibility testing of lactoferrin performed on 50 different strains of C. difficile.

46 of the 50 strains were inhibited by 4 mg/ml lactoferrin. No activity was demonstrated against one isolate at 16 mg/ml, and three isolates did not grow and were considered indeterminate.

### Example 8

### Control of C. difficile with Lactoferrin In Vivo

This example will be conducted to test the use of human lactoferrin derived from rice in the management of post-antibiotic colonization with C. difficile and the resulting inflammation reaction in the intestine in long-term care patients on enteral feeding.

Recombinant human lactoferrin will be tested in a specific population of patients receiving nutrition via an enteral feeding system. This population has been reported as having a higher susceptibility to C. difficile. Treatment will be initiated when broad spectrum antibiotic treatment is indicated and continued for eight weeks. Patients will be monitored for C. difficile by presence of a positive rapid ELISA for toxin A/B. All patients will be on enteral feeding by gastrostomy or jejunostomy tubes. The patients will receive either recombinant human lactoferrin from rice in 50 mM NaCl in enteral feeding flush (5 mg/mL lactoferrin in 600 mL of 0.3% saline administered every 24 hours of the 8 weeks of the study period) or 600 mL of 0.3% saline as a control.

It is expected that the population of patients who receive the lactoferrin treatment will have lower quantities of C. difficile when compared with the control population.

### Example 9

### Use of Lactoferrin to Treat Crohn's Disease

This study will be conducted to evaluate the effect on the Pediatric Crohn's Disease Activity Index (PCDAI) and physician's global assessment of recombinant human lactoferrin used as nutritional support in pediatric patients with active Crohn's disease.

The PCDAI is a multi-item measure that includes linear growth and places less emphasis on subjectively reported symptoms and more on laboratory parameters of intestinal inflammation. This index has been validated for use in children and adolescents and shown to discriminate between varying levels of disease activity. The PCDAI will be assessed at baseline, 4 weeks, 8 weeks and 12 weeks.

Recombinant human lactoferrin derived from rice is in a powder form. Both the treatment and placebo material will be provided in individual packets of 1 gram for reconstitution in juice or water. The suggested reconstitution will be in 250 mL, but this can be varied based on subject preference as long as the entire 1 gram dose is consumed at one sitting. The minimum reconstitution volume will be 150 mL. Dosing will be twice a day.

The study is expected to show that administration of recombinant human lactoferrin to children with mild to moderate Crohn's disease (new onset or recent flare) in addition to standard therapy will result in a faster stabilization of disease and/or improvement in disease as assessed by PCDAI and physician global assessment compared to standard therapy alone.

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

1. A method of preventing or treating an enteric disorder from occurring or recurring in a patient who has been exposed to an agent that causes enteric disorders, comprising the step of administering to said patient an oral formulation comprising milk protein.

2. The method of claim 1, wherein the enteric disorder is selected from the group consisting of cholera, diarrhea, cryptosporidiosis, foodborne disease, gastroenteritis, ulcers, inflammatory bowel disease, salmonellosis, typhoid and AIDS.

3. The method of claim 1, wherein the milk protein is isolated from milk or recombinantly-produced.

4. The method of claim 3, wherein the milk protein is isolated from milk derived from one or more human, bovine, porcine, and goat sources.

5. The method of claim 3, wherein the milk protein is produced recombinantly in plant cells.

6. The method of claim 4, where the milk protein is a recombinantly-produced human milk protein selected from the group consisting of lactoferrin, lysozyme, and combinations thereof.

7. The method of claim 1, wherein the oral formulation further comprises one or more additional proteins independently selected from the group consisting of defensins, cathelicidins, lactoferricin, and lactoperoxidase.

8. The method of claim 1, where the oral formulation is in the form of a solution.

9. The method of claim 1, where the oral formulation is in the form of a nutrition bar.

10. The method of claim 1, where the oral formulation is in the form of a powder suitable for reconstitution in water.

11. The method of claim 1, where the oral formulation is in the form of a pill, tablet or capsule.

12. The method of claim 1, where the agent that causes enteric disorders is selected from the group consisting of bacteria, viruses, fungi, and parasites.

13. The method of claim 12, where the bacteria are selected from the group consisting of Clostridium, Campylobacter, Salmonella, Shigella, and Escherichia.

14. The method of claim 12, where the viruses are selected from the group consisting of rotavirus, Norwalk virus, cytomegalovirus, herpes simplex virus, human immunodeficiency virus, and viral hepatitis.

15. The method of claim 12, where the parasites are selected from the group consisting of Giardia lamblia, Entamoeba histolytica, and Cryptosporidium.

16. A method of preventing and treating diarrhea from occurring or recurring in a patient who has been exposed to an agent that causes diarrhea, comprising the step of administering to said patient an oral formulation comprising recombinantly-produced human milk protein obtained from monocot seeds.

17. The method of claim 16, where the recombinantly-produced human milk protein is selected from the group consisting of lactoferrin, lysozyme, and combinations thereof.

18. The method of claim 16, wherein the oral formulation further comprises one or more additional proteins independently selected from the group consisting of defensins, cathelicidins and lactoperoxidase.

19. The method of claim 16, where said oral formulation is in the form of a solution.

20. The method of claim 16, where said oral formulation is in the form of a nutrition bar.

21. The method of claim 16, where the oral formulation is in the form of a powder suitable for reconstitution in water.

22. The method of claim 16, where the agent that causes diarrhea is an intestinal pathogen selected from the group consisting of bacteria, viruses, fungi, and parasites.

23. The method of claim 22, where the bacteria are selected from the group consisting of Clostridium, Campylobacter, Salmonella, Shigella, and Escherichia.

24. The method of claim 22, where the viruses are selected from the group consisting of rotavirus, Norwalk virus, cytomegalovirus, herpes simplex virus, and viral hepatitis.

25. The method of claim 22, where the parasites are selected from the group consisting of Giardia lamblia, Entamoeba histolytica, and Cryptosporidium

26. A method of preventing and treating inflammatory bowel disease in a patient suffering therefrom, comprising the step of administering to said patient an oral formulation comprising recombinantly-produced human milk protein obtained from monocot seeds.

27. The method of claim 26, where said inflammatory bowel disease is selected from the group consisting of Crohn's disease and ulcerative colitis.

28. The method of claim 26, where the recombinantly-produced human milk protein is selected from the group consisting of lactoferrin, lysozyme, and combinations thereof.

29. The method of claim 28, where the oral formulation further comprises one or more additional proteins independently selected from the group consisting of defensins, cathelicidins and lactoperoxidase.

30. The method of claim 26, where said oral formulation is in the form of a solution.

31. The method of claim 26, where said oral formulation is in the form of a nutrition bar.

32. The method of claim 26, where the oral formulation is in the form of a powder suitable for reconstitution in water.

33. A method of promoting growth of beneficial intestinal flora in a patient's digestive tract, comprising the step of administering to the patient an oral formulation comprising recombinantly-produced human milk protein obtained from monocot seeds.

34. The method of claim 33, where the recombinantly-produced human milk protein is selected from the group consisting of lactoferrin, lysozyme, and combinations thereof.

35. The method of claim 34, where the oral formulation further comprises one or more additional proteins independently selected from the group consisting of defensins, cathelicidins and lactoperoxidase.

36. The method of claim 33, where said oral formulation is in the form of a solution.

37. The method of claim 33, where said oral formulation is in the form of a nutrition bar.

38. The method of claim 33, where the oral formulation is in the form of a powder suitable for reconstitution in water.

39. An oral rehydration solution comprising:
recombinant human lactoferrin in an amount from about 0.5 to about 5.0 g/L; and
recombinant human lysozyme in an amount from about 0.1 to about 1.0 g/L, wherein the oral rehydration solution has an osmolality of from about 200 to about 310 mOsm/L.

40. The oral rehydration solution of claim 39, wherein the recombinant human lactoferrin and the recombinant human lysozyme are obtained from monocot seeds.

41. An oral rehydration formulation in the form of a powder, comprising recombinant human lactoferrin and recombinant human lysozyme, wherein, upon reconstitution with water to provide an oral rehydration solution, the oral rehydration solution provides:
recombinant human lactoferrin in an amount from about 0.5 to about 5.0 g/L; and
recombinant human lysozyme in an amount from about 0.1 to about 1.0 g/L, wherein the oral rehydration solution has an osmolality of from about 200 to about 310 mOsm/L.

42. The oral rehydration formulation of claim 41, wherein the recombinant human lactoferrin and the recombinant human lysozyme are obtained from monocot seeds.

43. An oral rehydration formulation in the form of a liquid concentrate, comprising recombinant human lactoferrin and recombinant human lysozyme, wherein, upon addition of water to provide an oral rehydration solution, the oral rehydration solution provides:
recombinant human lactoferrin in an amount from about 0.5 to about 5.0 g/L; and
recombinant human lysozyme in an amount from about 0.1 to about 1.0 g/L, wherein the oral rehydration solution has an osmolality of from about 200 to about 310 mOsm/L.

44. The oral rehydration formulation of claim 43, wherein the recombinant human lactoferrin and the recombinant human lysozyme are obtained from monocot seeds.

45. A method of treating diarrhea in a patient suffering therefrom, comprising the step of administering to said patient an oral rehydration solution according to claim 39.

46. A method of treating diarrhea in a patient suffering therefrom, comprising the step of administering to said patient an oral rehydration solution according to claim 41.

47. A method of treating diarrhea in a patient suffering therefrom, comprising the step of administering to said patient an oral rehydration solution according to claim 43.
